(19) United States
(12) Patent Application Publication
(10) Pub. No.: US 2019/0144910 A1
(43) Pub. Date: May 16, 2019

(54) PRODUCT DEMONSTRATION DEVICE

(71) Applicant: The Procter & Gamble Company, Cincinnati, OH (US)

(72) Inventors: Yang SU, Beijing (CN); Lei TIAN, Beijing (CN); Yumeng OUYANG, Beijing (CN)

(21) Appl. No.: 16/184,587

(22) Filed: Nov. 8, 2018

(30) Foreign Application Priority Data
Nov. 16, 2017 (CN) 201721532586.1
Nov. 16, 2017 (WO) CN2017/111250

(51) Int. Cl. C12Q 1/02; B01L 3/00

(52) U.S. Cl. CPC C12Q 1/025; B01L 3/50; B01L 2300/18; B01L 2300/021; B01L 2300/027; B01L 2200/16

(57) ABSTRACT

A device that conducts side-by-side demonstration of products helps an audience to understand the product benefits of one product over another product.

### FIELD OF THE INVENTION

The present invention relates to a product demonstration device useful for demonstrating a product benefit.

### BACKGROUND OF THE INVENTION

Product demonstrations are an important means of demonstrating product benefits to potential customers and users. However, some of the challenges include one or more of the following: need for scientific training to conduct the demonstration, need to use expensive or complex scientific equipment, need for portability of the demonstration (set-up/take-down time and transportation challenges), need to minimize the time it takes to conduct demonstration (i.e., minimizing duration so that the attention of audience can best be captured), need to minimize the time it takes to prepare for the next demonstration (i.e., minimizing delay from one demonstration to the next), need for safety in using the device (and handling reactants), and need to manufacture the device relatively economically. Accordingly, there is an opportunity to provide a product demonstration that meets one or more of these needs, especially in oral care products where products benefits are sometimes difficult to visualize/understand by audiences.

### SUMMARY OF THE INVENTION

The present invention is based, in part, upon the discovery of a compact and portable device that automates at least some of the steps of the product demonstration while still allowing the audience (e.g., customers and users) to view the demonstration. The device also helps to facilitate a side-by-side demonstration of the test product and the control product.

One aspect of the invention provides a product demonstration device, preferably that is compact or portable, more preferably compact and portable, capable of demonstrating, preferably demonstrating concurrently and repeatedly, a product benefit from a test product compared to a control product. The device comprises: a liquid container preferably having a volumetric capacity from 20 ml to 2,000 ml; a test reaction vessel in fluid communication with the liquid container, preferably having a volumetric capacity from 1 ml to 500 ml; and a control reaction vessel in fluid communication with the liquid container, preferably having a volumetric capacity from 1 ml to 500 ml. A first motorized mixer is functionally attached to the test reaction vessel (capable of mixing the interior space of the test reaction vessel). A second motorized mixer is functionally attached to the control reaction vessel (capable of mixing the interior space of the control reaction vessel). A first dispensing port is in fluid communication with the test reaction vessel, and capable of receiving: chemical or biological reactants, and a test product. A second dispensing port is in fluid communication with the control reaction vessel, and capable of receiving: chemical or biological reactants, and a control product. A first reaction in the test reaction vessel amongst the mixed chemical or biological reactants and test product is compared to a second reaction in the control reaction vessel amongst the mixed chemical or biological reactants and the control product to demonstrate the product benefit of the test product. A non-limiting example of a product benefit is anti-microbial benefit. A non-limiting example of a product is toothpaste.

Another aspect of the invention provides for a kit that comprising this device and optional usage instructions.

Another aspect of the invention provides for a method of conducting a product demonstration demonstrating a test product providing a product benefit compared to a control product comprising the use of the aforementioned device or kit.

Another aspect of the invention provides for a method of determining whether an oral care product inhibits hydrogen sulfide production comprising the steps: mixing a solution of yeast, cysteine, and oral care product; and assessing a reaction of the mixed solution to determine whether the oral care product inhibits hydrosulfide production. Preferably the oral care product is a toothpaste.

While the specification concludes with claims that particularly point out and distinctly claim the invention, it is believed the present invention will be better understood from the following description.

### BRIEF DESCRIPTION OF THE DRAWINGS

The embodiments set forth in the drawings are illustrative in nature and not intended to limit the invention defined by the claims. The following detailed description of the illustrative embodiments can be understood when read in conjunction with the following drawings, where like structure is indicated with like reference numerals and in which:

FIG. 1 is a front view of a compact and portable product demonstration device;

FIG. 2 is a front view of the device of FIG. 1, wherein certain components are shown in an exploded view; and

FIG. 3 is a perspective view of the device of FIGS. 1 and 2, with many of the components, especially on the left side, are shown in an exploded view.

### DETAILED DESCRIPTION OF THE INVENTION

The following text sets forth a broad description of numerous different embodiments of the present disclosure. The description is to be construed as exemplary only and does not describe every possible embodiment since describing every possible embodiment would be impractical, if not impossible. It will be understood that any feature, characteristic, component, composition, ingredient, product, step or methodology described herein can be deleted, combined with or substituted for, in whole or part, any other feature, characteristic, component, composition, ingredient, product, step or methodology described herein. Numerous alternative embodiments could be implemented, using either current technology or technology developed after the filing date of this patent, which would still fall within the scope of the claims.

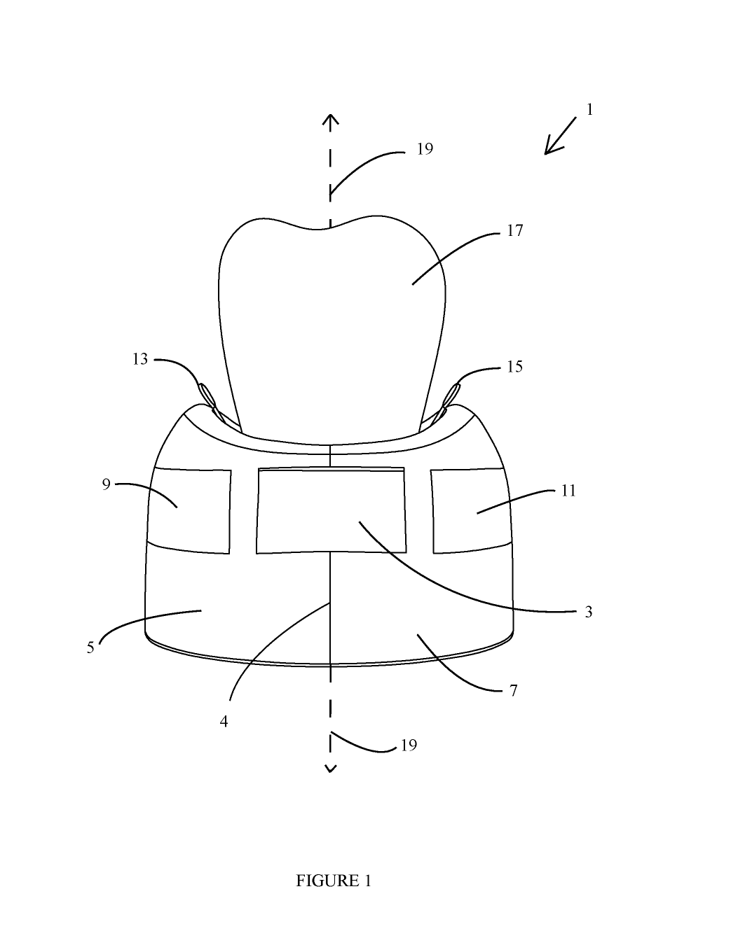

FIG. 1 is a front view of a non-limiting example of a compact product demonstration device (1) of the present invention. Preferably the device (1) is directed to oral care products, more preferably wherein the oral care products are dentifrice products, especially toothpaste. The device (1) has a prominent tooth shaped cover (17) at the upper most section of the device (1) and generally located centrally in the device (1) aligned along the longitudinal axis (19). Preferably the tooth shaped cover (17) is made from plastic, more preferably having a white color, and has an area (when removed from the device (1)) from 100 cm³ to 50,000 cm³, more preferably 1,000 cm³ to 20,000 cm³, yet more preferably from 2,000 cm³ to 4,000 cm³. The tooth shaped cover (17) indicates to the audience that the product demonstration is directed to oral care products. Of course, other objects can be substituted for a tooth depending upon the product being demonstrated. The device (1) generally has bilateral symmetry along the longitudinal axis (19). Left and right panels (5, 7) are on opposing sides of the device (1) come together to form a seam (4) (on the front side of the device (1)). There is also a seam on the back side of the device (not shown) of the device (1). Of course, a no seam option is also possible if the panels are made as a unitary piece. A plane intersecting the seam (4) of the front of the device (1), longitudinal axis (19), and seam on the back side of the device (1) divides the device into essentially two halves with bilateral symmetry to each other. In other words, the device has bilateral symmetry (save minor inconsequential differences). One advantage of having bilateral symmetry is that the audience is provided implicit messaging that the control and test products are treated the same. Preferably the demonstration can generally be observed from a single viewing point. On opposing sides of the device (1) there are left and right engageable testing receivers (13, 15, respectively) and left and right window covers (9, 11 respectively). There is also a control module (3) located centrally on the front side of the device (1). The control module (3) allows the user to select certain functions of the device. Preferably the control module (3) is a touch screen to provide convenience to the operator and imply to audience members the sophistication of the device.

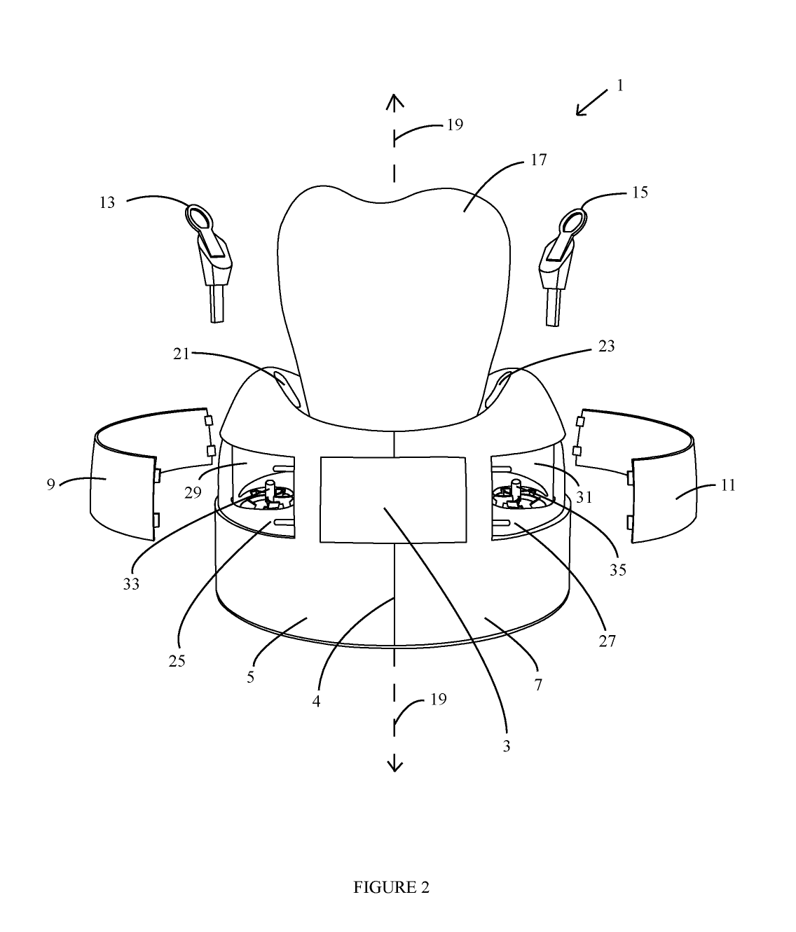

FIG. 2 is also a front view of the compact product demonstration device (1) (as shown in FIG. 1), but with exploded views: of the left and right window covers (9, 11, respectively) removed; and the left and right engageable testing receivers (13, 15, respectively) disengaged. The left and right window covers (9,11) provide the function of covering the left and right windows (25, 27, respectively) protecting the windows, when the device (1) is not in use, from scratching or damage (e.g., during transport etc.). This is particularly important for salesperson operators who often travel from location to location (to conduct such product demonstrations). Through the respective windows (25, 27), the left and right reaction vessels (29, 31, respectively) can be visually observed. These reaction vessels (29, 31) have side walls where at least a portion is made of a clear or transparent material, preferably a clear material, more preferably clear plastic, so that mixing/reactions contained within the interior space of the reaction vessels (29, 31) are observable by the audience (and through the windows (25, 27)). The windows (25, 27) are “cut-out” from the respective left and right panels (5, 7). These panels (5,7) are preferably made from plastic, more preferably pink or red in color, or other color representative of human gums. The color of the left and right panels (5,7) represent gums and windows into the panels indicates to the audience that the reaction is happening at or below the gum line (at the intersection of the gum and tooth). Of course the panels can be in another configuration to compliment the type of product being demonstrated.

Still referencing FIG. 2, the left and right engageable testing receivers (13, 15, respectively) releasably engage the respective left and right dispensing ports (21, 23, respectively). In turn, the dispensing ports (21, 23) are in fluid communication with the respective reaction vessels (29, 31). These dispensing ports (21, 23) are used by the operator to dispense chemical or biological reactants and/or products. Although not shown, this dispensing can be conducted manually by the operator or even automatically (e.g., by the device itself or a machine external to the device).

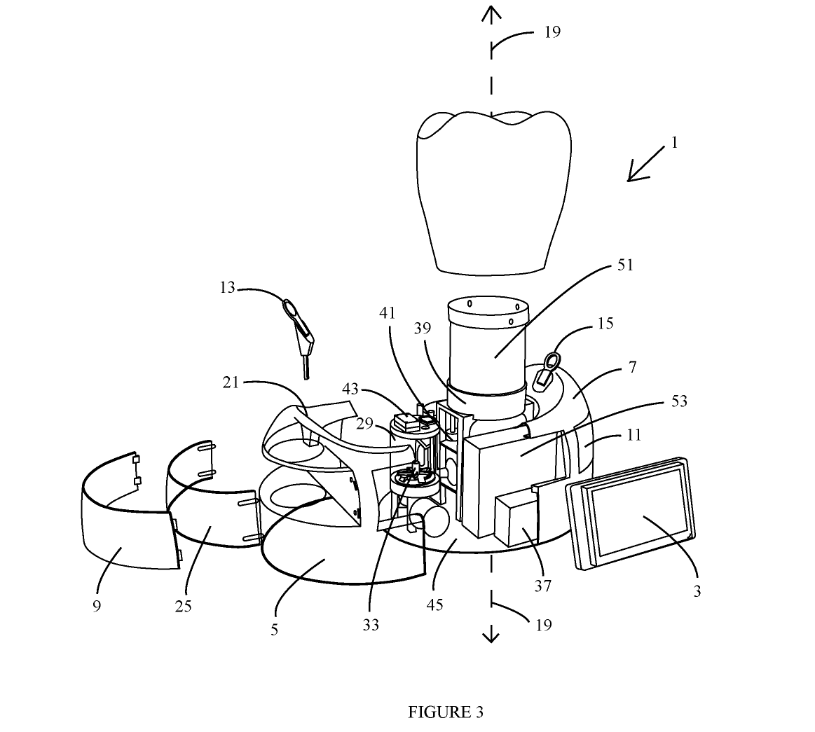

FIG. 3 is a perspective front view of the product demonstration device (1) with certain components of the left side shown in an exploded view. The tooth shaped cover (17) is releasably affixed to the device so the operator can remove the cover (17) to refill the liquid container (51) with liquid. The tooth shaped cover (17) is shown exploded elevated along the longitudinal axis (19). Preferably the liquid container (51) has a volumetric capacity from 20 ml to 2,000 ml, preferably 200 ml to 1,000 ml, more preferably 400 ml to 800 ml of liquid. Liquid (not shown) is contained in the liquid container (51) to conduct product demonstrations, preferably conduct multiple product demonstrations as well as clean the reaction vessels (29, 31) in-between each product demonstration. The liquid may be simply water (which is advantageous for cost and availability reasons). Distilled or otherwise purified water (to help with the integrity of the experiment) can also be used. Alternatively, the liquid may be pH buffered or contain salts (e.g., saline solution), preferably saline and pH conditions (or other conditions) mimicking human saliva or human oral cavity. Alternatively, the liquid mimics the conditions of the product's intended use (e.g., laundry cleaning). Preferably a heating element (not shown) is in thermal communication with the liquid contained in the liquid container (51). The heating element heats the liquid so that the reaction by the product demonstration occurs more rapidly (i.e., reactants react more rapidly) or helps with the integrity of the experiment (by mimicking the temperature of the oral cavity or otherwise conditions of the intended use of the product). A temperature control unit (39) is in electrical communication with the heating element, which controls the temperature of the contained liquid heated from the heating element. Preferably the temperature of the liquid is from 20° C. to 100° C., more preferably from 40° to 100° C., yet more preferably from 60° C. to 90° C.; alternatively, from 35° C. to 40° C.

Still referring to FIG. 3, the heated liquid (contained in the liquid container (51)) is dispensed in equal portions to the left and right reaction vessels (29, 31). The dispensing can by way of a pump (41) or by gravity (or both). The pump (41) is in fluid communication with the contained liquid and reaction vessels (29, 31) by way of a hose or tube etc. (not shown). The liquid container (51) is in the device (1) in a position higher in elevation (with respective to the longitudinal axis (19)) as compared to the reaction vessels (29, 31), wherein the reaction vessels (29, 31) are aligned at same elevation. From 5 ml to 100 ml, preferably from 10 ml to 50 ml, more preferably from 15 ml to 30 ml, of the liquid, preferably heated liquid, are dispensed to each of the reaction vessels (29, 31). The test reaction vessel (29) has a volumetric capacity from 1 ml to 500 ml, preferably from 50 ml to 400 ml, more preferably from 100 ml to 300 ml, and is in fluid communication with the liquid container. The control reaction vessel (31) has a volumetric capacity from 1 ml to 500 ml, preferably from 50 ml to 400 ml, more preferably from 100 ml to 300 ml, and is in fluid communication with the liquid container. Preferably the test reaction vessel (29) and the control reaction vessel (31) have the same volumetric capacity (in the interests of keeping the variables between the two vessels the same).

Independently either before or after the liquid dispensed, chemical or biological reactions are dispensed into each of the reaction vessels (29, 31). In one example, the product benefit is an anti-hydrogen sulfide benefit. Hydrogen sulfide is an undesirable result metabolic activity of microbes in the oral cavity. It is undesirable because of the unpleasant odor, i.e., bad breath. It can also act as a proxy for an anti-microbial product benefit, given that a gingival sulcus sulfur test is one of the dental clinical approaches to determine subgingival microbial activity. It is advantageous to have an oral care product that prevents, or at least mitigates against, hydrogen sulfide generation (by microbes). To demonstrate this product benefit, the biological reactant of yeast is used (to mimic microbes in the oral cavity) and the chemical reactant of cysteine is used (a sulfur containing amino acid to mimic food residence in the oral cavity). Preferably the yeast is baker's yeast. An example of a unit dose of yeast and cysteine is 0.1 grams to 10 grams of yeast and from 0.1 gram to 1 gram of cysteine (to be dispensed in each reaction vessel (29, 31)). The operator dispenses these biological and chemical reactants into the respective reaction vessels (29, 31) through the dispensing ports (21, 23) (in equal portions). In one example, the chemical reactant and/or biological reactant or combined in a single unit dose, preferably in a water-soluble unit dose so that demonstration is conducted quickly and accurately. The water-soluble unit dose encapsulates a pre-measured amount of the reactant(s) in a water-soluble film, e.g., polyvinyl alcohol film. The operator also dispenses the oral care products (i.e., a control product and test product) into the reaction vessels (29, 31) via the dispensing ports (21, 23). If the product is toothpaste, it can be dispensed via an injector such as syringe. The amount of product dispensed may be from 0.1 gram to 20 grams, preferable from 1 to 10 grams, more preferably from 1 to 5 grams, into the interior space of each reaction vessel (29, 31). The product demonstration of course need not be limited to oral care products. Rather, a broad range of products may be used including hand, body, hair soaps and shampoos, or home cleaning products, or laundry detergents and the like. A broad range of product benefits may also be demonstrated including anti-microbial, anti-acid, anti-toxin (e.g., bacterial lipopolysaccharide so called “LPS”) benefits. The products may be solid, liquid, semi-solid, semi-liquid, or combinations thereof.

Each reaction vessel (29, 31) has a left and right motorized mixer (33, 35, respectively) in fluid communication with the contents contained in the reaction vessel (29, 31) (i.e., interior space of the reaction vessel (29, 31)). The motorized mixer (33, 35) provides advantages including quickly and thoroughly mixing the dispensed chemical and biological reactants as well as the dispensed products so that the time of the demonstration is reduced. Moreover, if the product (or reactants for that matter) is viscous or otherwise challenging to quickly disperse within the reaction vessel, the motorized mixer (33, 35) helps to solubilize or otherwise disperse the product (to mimic the product intended use conditions (e.g., brushing teeth with toothpaste)). The motorized mixer (33, 35) may have rotations per minute (RPM) values from 1 RPM to 50,000 RPM, preferably from 2o RPM to 20,000 RPM, more preferably from 500 RPM to 100 RPM. Preferably the motorized mixer (33, 35) mixes the contents contained within the reaction vessel from 1 second to 60 seconds, preferably from 3 seconds to 30 seconds, more preferably from 5 seconds to 25 seconds, yet more preferably from 10 seconds to 20 seconds. Examples of viscous products, such as toothpaste, have a viscosity range from 150,000 centipoises to 850,000 centipoises (“cP”). A method for assessing viscosity is by way of a viscometer such as Brookfield® viscometer.

The results of the reaction can be assessed by a sensor (43) or manually by way of a disposable test strip releasable received in the engageable testing receiver (13, 15). For example, the test strip is chemically treated paper that turns black to indicate the presence of hydrogen sulfide, otherwise the paper remains a white color to indicate no hydrogen sulfide present. The test strip is replaced after each hydrogen sulfide assessment. It provides the advantage of being inexpensive and easily understood by the audience. Alternatively, or in addition, the sensor is employed that automatically sensing the presence (and even amount) of hydrogen sulfide or other proxy of the demonstrable consumer benefit. The sensor is in electrical communication to an audible or visual signal so the audience will readily and timely appreciate the results of the sensor (and which reaction vessel (29, 31) the results are directed to).

In another example, the product benefit of the demonstration is as an anti-acid. The chemical reactants are glucose or sucrose, and the biological reactants are acid-generating microbes. Briefly when the sensor or the test strip senses pH decrease, it is a proxy of the demonstrated consumer anti-cavity benefit of the product. Preferably, the product in this demonstration is a toothpaste, for example a sodium fluoride or sodium monofluorophosphate containing toothpaste.

In yet another example, the product benefit of the demonstration is as an anti-toxin. The reactants are endotoxin-generating microbes and Limulus Amebocyte Lysate reagent. Briefly, the resulting reaction solution changes from colorless to yellowish depending on the absence or the presence of endotoxin. The results of the color change reaction can be observed through the windows (25, 27) directly, preferably observed after addition of diazotization staining agent to the solution to enhance the contrast. It is a proxy of the demonstrated anti-toxin benefit of the product. Preferably, the product in this demonstration is an anti-toxin toothpaste or mouthwash.

In yet another example, the product benefit of the demonstration is as an anti-bacterial. The biological reactants are Staphylococcus aureus, Escherichia coli, Candida albicans, or any combination thereof. Briefly, when addition of bacteria activity assaying reagent (such as Soler™) into the reaction, the color of solution changes depending on the activity of bacteria. The results of the color change can be observed through the windows (25, 27). It is a proxy of the demonstrated anti-bacteria benefit of the product. Preferably, the product in this demonstration is an anti-bacterial soap, bar soap, liquid soap, detergent, laundry detergent, floor cleaner, dish detergent, shampoo, hair shampoo, or body shampoo.

In yet another example, the product benefit of the demonstration is deep cleaning. The reactants are the lipid stains deep in the gum sulcus or on the surface of teeth and surfactant in the product. Briefly, the surfactant is amphiphilic with both hydrophobic groups and hydrophilic groups. Therefore, it can react with lipid stains and bond together and then dissolve in the water. It is a proxy of the demonstrated cleaning benefit of the product. Preferably, the product in this demonstration is an anti-bacterial soap, bar soap, liquid soap, detergent, laundry detergent, floor cleaner, dish detergent, shampoo, hair shampoo, or body shampoo.

After the conclusion of the demonstration, the device can prepare itself automatically for the next demonstration (i.e., initiating a wash cycle) by evacuating the spent reactants and spent products into a waste container (not shown); and thereafter washing the reaction vessels by dispensing liquid contained in the liquid container (51) into the reaction vessel and mixing the liquid therein by the motorized mixer (33, 35). Lastly, the spent wash liquid is also evacuated into the waste container. The waste container may be either integral or external to the device (1). The waste container and reaction vessels (29, 31) are in fluid communication with each other. Contents of the reaction vessels (29, 31) are dispensed to the waste container by gravity and/or via the pump (41) (or both). In one preferred example, the waste container (independent of the device) is vertically below the reaction vessels (29, 31) to have gravity at least assist in the evacuation. One advantage of having the waste container independent of the device (1) is to improve the portability of it by keeping the device smaller in size, simpler in construction, and arguably faster in recharging the device for another series of demonstrations. In another example, the waste container is a drain basin. Alternatively, there is not waster container but rather the device is directly and fluidly connected to a drain. The wash cycle typically uses from 5 ml to 200 ml, preferably from 10 to 80 ml, more preferably from 20 to 40 ml of liquid (that is contained in the liquid container (51)).

One advantage of the device is the relative speed that a single demonstration is conducted, wherein preferably the demonstration time is from 10 seconds to 180 seconds, preferably from 15 seconds to 120 seconds, alternatively from 30 seconds to 90 seconds, from the initiation of the demonstration (e.g., liquid from the liquid container dispensed into the reaction vessel) to the conclusion of the demonstration (e.g., signal form the sensor or visible result from the test strip).

Another advantage of the device is the relative speed that washings in-between each demonstration is conducted, wherein the preferably washing time is from 10 seconds to 180 seconds, preferably from 20 seconds to 120 seconds, alternatively, from 30 seconds to 60 seconds. The start of the washing cycle commencing once the spent contents contained in the reaction vessels (29, 31) begin to evacuate from the reaction vessels (to the waste container) and are completed once the device is ready to begin the next demonstration.

Yet another advantage is the number of demonstrations in a single charge of the device (before having to refill the liquid in the liquid container etc.). Preferably the device conducts 2 to 20 demonstrations including washings in-between, more preferably 3 to 10, yet more preferably from 3 to 5. This is notable given the relatively small size and mass of the device. To this end, the device is preferably from 5,000 cm³ to 150,000 cm³, more preferably 10,000 cm³ to 100,000 cm³, yet more preferably from 30,000 cm³ to 50,000 cm³. The mass of the device, without any consumables (e.g., empty, without liquid contained in the liquid container) is from 0.1 kg, to 50 kg, preferably from 0.1 kg to 20 kg, yet more preferably from 0.1 kg to 10 kg. This small size and mass provides portability and/or compactness that is important to the operator (e.g., traveling salesperson).

Referring to FIG. 3, the base (45) forms the bottom of the device (1) and opposes the tooth shaped cover (17) (relative to the longitudinal axis (19)). The base (45) is a regular oval shape, wherein the longitudinal axis (19) passes through the center thereof. The different components of the device (1) are affixed, directly or indirectly, to the base. Preferably the base is made from metal to allow for sufficient strength to provide support for the device as a whole. The left and right panels (5, 7) generally mimic the regular oval shape.

Many components of the device are in electrical communication with each other. FIG. 3 provides a central processing unit (CPU) (37) that is in electrical communication with the battery (53) (as an energy source), control module (3) (providing an interface to the operator), pump (41), motorized mixer (33, 35), and temperature control unit (39), valves (not shown) (to open and close access to and from reaction vessels (29, 31)). The battery (53) helps with portability (by not needing the device (1) in the proximity of a mounted electrical socket during operation). Of course, alternatively the device (1) may be engageable with an electrical socket for not only recharging the battery (53) but also simply operating the device (1) without the use of a battery.

One aspect of the invention provides for a kit comprising: a product demonstration device (as described herein); unit doses of chemical or biological reactants; a unit dose of a test product; and a unit dose of a control product. Disposable, (i.e., one time use) test strips are optional, but preferred. Usage instructions (for the kit) is optional but preferred. The term “unit dose” means the appropriate amount of the reactant to conduct the demonstration under the subject conditions. Preferably the unit dose is pre-measured, even pre-packaged, before the demonstration is initiated to minimize time in conducting the demonstration or plurality of demonstrations. The unit dose can take the form of a water-soluble unit dose. For example, a water-soluble film (e.g., made from a polyvinyl alcohol) may encapsulate one or more the reactants (e.g., sachet) so the user can simply dose the unit dose in the reaction vessels (via the respective dispensing ports). Alternatively, the sachet is not water soluble and the user opens the sachet and empties the pre-measured contents into the device. In one example, the chemical reactant is cysteine and the biological reactant is yeast. Preferably the unit dose of yeast is from 0.1 gram to 10 gram, preferably from 1 gram to 5 gram. Preferably the unit dose of cysteine is from 0.01 gram to 1 gram, preferably from 0.1 gram to 0.5 gram. Preferably the unit dose of product is from 0.1 gram to 10 gram, preferably from 1 to 5 gram. More preferably the product is an oral care product, even more preferably toothpaste. Preferably the unit dose of chemical and/or biological reactants are combined as to simplify and expedite the demonstration. Preferably the product, especially of relatively high viscosity, is dispensed into the interior space of the reaction vessels by injecting the product via an injector (via the dispensing ports). More preferably a plurality of unit doses of the reactants and products are provided to facilitate conducting a plurality of demonstrations (as to provide the advantage of minimizing time in-between demonstrations). Preferably the testing strips are disposable paper testing strip that turn a color upon the presence of a reactant ingredients, such as turning black (i.e., a non-white color) in the presence of a hydrogen sulfide.

Another aspect of the invention provides for a method of conducting a product demonstration demonstrating a test product providing a product benefit compared to a control product comprising the following steps. A first step is providing a portable product demonstration device or kit as herein described. A liquid contained in the liquid container is dispensed into the test and control reaction vessels. Chemical and/or biological reactants are dispensed into the test and control reaction vessels via the first and second dispensing ports, respectively. A test product is dispensed into the test reaction vessel via the first dispensing port; and a control product is dispensed into the control reaction vessel via the second dispensing port. Contents contained in the test and control reaction vessels are mixed by first and second motorized mixers, respectively. Lastly, the resulting reaction of the mixed contents contained in the test and control reaction vessels are assessed to demonstrate the product benefit of the test product compared to the control product.

Preferably the method is conducted in less than 180 seconds, preferably the method further comprising a washing cycle conducted in less than 240 seconds; more preferably the method further comprising a second demonstration, and wherein the first demonstration, wash cycle, and second demonstration are conducted in less than 600 seconds.

Another aspect of the invention provides a method of determining whether an oral care product inhibits hydrogen sulfide production comprising the steps: mixing a solution of yeast, cysteine, and oral care product; and assessing a reaction of the mixed solution to determine whether the oral care product inhibits hydrosulfide production. Preferably the solution comprises: 1% to 50%, preferably 5% to 15%, by weight of the solution of yeast; 0.01% to 5%, preferably 0.05% to 0.15%, by weight of the solution of cysteine; 1% to 50%, preferably 20% to 40%, by weight of the solution of oral care product. Preferably the reaction is assessed by either: a hydrogen sulfide sensor in fluid communication with the aforementioned solution; or a testing strip capable of changing color upon the presence hydrogen sulfide in fluid communication with the aforementioned solution.

### Example

A stannous containing toothpaste is demonstrated to have anti-microbial activity (as a product benefit) by inhibiting baker's yeast's ability to metabolize cysteine given the lack of hydrogen sulfide production (that is otherwise comparatively shown by the control toothpaste that does not contain stannous). It is the stannous that provides this product benefit. A device as described in FIGS. 1-3 is manufactured by Tianjin Hope Biotech Co. Ltd, Tianjin, China. Stannous containing toothpaste is the test product while a non-stannous containing toothpaste is the control product. A unit dose of each toothpaste (3 grams) is prepared in respective injectors. Unit doses of dry baker's yeast (3 grams; S. cerevisiae) (as the biological reactant) and cysteine (0.25 grams; CAS No.: 52-90-4) (as the chemical reactant) are combined and prepared in small water soluble sachets. The water container of the device is filled with water. A “warm” button is pressed on the touch screen control module for the heating element to heat the water (contained in the water container) to achieve a desired temperature of 80 degrees Celsius. The touch screen control module indicates “Ready!” when the water achieves the desired temperature. A “water pumping” button is pressed to have the device concurrently dispense 20 ml of the heated water each into the test and control reaction vessels. The first and second engageable testing receivers are removed from the respective first and second dispensing ports thereby providing access to the interior space of test and control vessels. Unit doses of the biological and chemical reactants are dispensed into the test and control reaction vessels through the respective dispensing ports. The engageable testing receivers are reinserted into the dispensing ports. A “mix” button is pressed on the module to concurrently mix the respective reaction vessels, wherein each of the motorized mixers operates for 45 seconds at 300 RPM. The mixed reactants are allowed to react from 30 seconds to 60 seconds. Thereafter, hydrogen sulfide sensors are engaged to assess that indeed hydrogen sulfide is being produced (by the yeast metabolizing the cysteine) and detectable within the interior space of the reaction vessels. A “detect” button is pressed to switch on/off the illustration of hydrogen sulfide level the on the module. Alternatively, disposable paper testing strips that turn black (from a white color) upon the exposure of hydrogen sulfide are employed and functionally releasably attached to the engageable testing receives to assess the hydrogen sulfide production within the interior space of the reaction vessels.

After demonstrating hydrogen sulfide production, the engageable testing receivers are removed from the dispensing ports to allow contained hydrogen sulfide to escape from the reaction vessels (via the dispensing ports) for about 30 seconds. Thereafter, the unit dose of the stannous toothpaste is injected through the first dispensing port into the test reaction vessel, and the unit dose of the non-stannous toothpaste is injected through the second dispensing port in into the control reaction vessel. The engageable testing receivers are reinserted into the dispensing ports and the “mix” button is once again pressed on the module to mix the respective reaction vessels, wherein each of the motorized mixers operate for about 30 seconds at 300 RPM. The engageable testing receivers have the spent testing strips removed and replaced with new testing strips and then reinserted into the dispensing ports. After about 30 seconds, results from the testing strips are assessed. The more the white color remains on the testing strip, the more successful the toothpaste is inhibiting hydrosulfide production and thus the greater the antimicrobial benefit. Said in another way, the darker or more black the testing strip, the less successful the toothpaste is inhibiting hydrosulfide production and thus the less antimicrobial benefit (if any). Accordingly, the stannous containing toothpaste (i.e., test product) results in a testing strip that is more white in color (i.e., less black) compared to the non-stannous toothpaste (i.e., control product). Without wishing to be bound by theory, the stannous inhibits the growth and metabolism of the baker's yeast as to prevent the baker's yeast from metabolizing the cysteine (and thus no hydrosulfide is produced). In contrast, the non-stannous toothpaste is not as effective inhibiting the growth and metabolism of the baker's yeast thereby allowing the baker's yeast to metabolize the cysteine thereby emitting hydrosulfide which is detectable by the disposable paper test strip turning black (from its starting white color). The entire demonstration takes less than 180 seconds to conduct (not including the time it takes to prepare unit doses of the reactants and products).

The device is prepared for the next demonstration by two steps. Firstly, a “drain” button is pressed for the device to empty the spent contents of the reaction vessels to a waster container. Secondly, a “wash” button is pressed to have the device dispense 20 ml of heated water to the reaction vessels (from the liquid container) and engage the motorize mixers for 5 seconds at 300 RPM, and then finally evacuate the spent wash contents to the waste container. After visual inspection of the reaction vessels, the wash button may be pressed again as needed. The device is now ready for another demonstration. The device is prepared in less than 30 seconds according to these two steps (assuming a single wash cycle).

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A product demonstration device (1) capable of demonstrating a product benefit from a test product compared to a control product, wherein the device comprises:
a) a liquid container (51) having a volumetric capacity from 20 ml to 2000 ml;
b) a test reaction vessel (29) having a volumetric capacity from 1 ml to 500 ml, in fluid communication with the liquid container (51);
c) a control reaction vessel (31) having a volumetric capacity from 1 ml to 500 ml, is in fluid communication with the liquid container (51);
d) a first motorized mixer (33) functionally attached to the test reaction vessel (29);
e) a second motorized mixer (35) functionally attached to the control reaction vessel (31);
f) a first dispensing port (21) in fluid communication with the test reaction vessel (29) capable of receiving: chemical or biological reactants, and a test product; and
g) a second dispensing port (23) in fluid communication with the control reaction vessel (31) capable of receiving: chemical or biological reactants, and a control product.

2. The portable product demonstration device (1) of claim 1, wherein:
a) the liquid container (51) having a volumetric capacity from 200 ml to 1000 ml, preferably from 400 ml to 800 ml;
b) the test reaction vessel (29) having a volumetric capacity from 50 ml to 400 ml, preferably from 100 ml to 300 ml;
c) the control reaction vessel (31) having a volumetric capacity from 50 to ml 400 ml, preferably from 100 ml to 300 ml.

3. The portable product demonstration device (1) of claim 1 or 2, wherein at least a portion of the interior space of the test and control reaction vessels (29, 31) are each visible externally from the device (1); preferably wherein the volumetric capacity of the test and control reaction vessels are the same (29, 31); more preferably wherein test and control reaction vessels (29, 31) are on opposing sides of the device (1); yet more preferably the device (1) is bilaterally symmetrical.

4. The portable product demonstration device (1) of any one of the preceding claims, further comprising a heating element (not shown) in thermal communication with (contents of) the liquid container (51).

5. The portable product demonstration device (1) of any one of the preceding claims, further comprising a pump (41) in fluid communication with the liquid container (51) and to the test and control reaction vessels (29, 31).

6. The portable product demonstration device (1) of any one of the preceding claims, further comprising: a first engageable testing receiver (13) releasably engageable with the first dispensing port (21); and a second engageable testing receiver (15) releasably engageable with the second dispensing port (23).

7. The portable product demonstration device (1) of claim 6, wherein the first and second engageable testing receivers (13, 15) are each capable of engaging a disposable test strip (not shown).

8. The portable product demonstration device (1) of any one of the preceding claims, further comprising a central processing unit (37) and a control module (3), wherein the central processing unit (37) is in electrical communication with at least: the control module (3); and first and second motorized mixers (33, 35); preferably the control module (3) is a touch screen control module (3).

9. The portable product demonstration device (1) of any one of the preceding claims, having a mass, devoid of any consumables, from 0.1 kg to 50 kg, preferably from 0.1 kg to 20 kg, more preferably from 0.1 kg to 10 kg.

10. The portable product demonstration device (1) of any one of the preceding claims, wherein the device is a portable oral care product demonstration device, preferably a portable toothpaste demonstration device; preferably the product benefit is an anti-microbial product benefit.

11. The portable product demonstration device (1) of any one of the preceding claims, further comprising a releasably received tooth shaped cover (17), preferably the tooth shaped cover (17) is releasably received over the liquid container (51) covering the liquid container; more preferably wherein the tooth shaped cover (17) is made from plastic, more preferably made from plastic colored white on at least the exterior surface; yet more preferably the tooth shaped cover (17) has an area from 100 cm³ to 50,000 cm³, preferably from 1,000 cm³ to 10,000 cm³, more preferably from 2,000 cm³ to 4,000 cm³.

12. A kit comprising:
a) a portable product demonstration device (1) of any one of the preceding claims;
b) a least two unit doses, preferably a plurality of unit doses, of yeast and cysteine;
c) at least one unit dose, preferably a plurality of unit doses, of a test oral care product; and
d) at least one unit dose, preferably a plurality of unit doses, of a control oral care product.
e) optionally at least two disposable test strips, preferably a plurality of test strips, wherein each of the test strips is capable of changing color in the presence of hydrogen sulfide; and
f) optionally usage instructions.

13. A method of conducting a product demonstration demonstrating a test product providing a product benefit compared to a control product comprising the steps:
a) providing a portable product demonstration device (1) according to any one of claims 1-11 or a kit according to claim 12;
b) dispensing a liquid contained in the liquid container into the control and reaction vessels (29, 31);
c) dispensing chemical or biological reactants into the test and control reaction vessels (29, 31), via the first and second dispensing ports (21, 23), respectively;
d) dispensing a test product into the test reaction vessel (29) via the first dispensing port (21) and a control product into the control reaction vessel (31) via the second dispensing port (23);
e) mixing contents contained in the control and reaction vessels (29, 31) via the first and second motorized mixers (33, 35), respectively; and
f) comparing a first reaction in the test reaction vessel (29) amongst the mixed chemical or biological reactants and test product to a second reaction in the control reaction vessel (31) amongst the mixed chemical or biological reactants and the control product, to demonstrate the product benefit of the test product.

14. The method of claim 13, further comprising heating the liquid contained in the liquid container (51) before dispensing to the reaction vessels (29, 31), preferably heating the liquid from 30° C. to 100° C.; more preferably concurrently dispensing the heated liquid to the reaction and control vessels (29, 31); yet more preferably concurrently dispensing from 5 ml to 100 ml, preferably from 10 ml to 50 ml, of the heated liquid;
preferably wherein the method is conducted in less than 180 seconds, preferably the method further comprising a washing cycle conducted in less than 240 seconds; more preferably the method further comprising a second demonstration, and wherein the first demonstration, wash cycle, and second demonstration are conducted in less than 600 seconds;
more preferably wherein the motorized mixers mix at 20 RPM to 20,000 RPM, more preferably from 500 RPM to 1,000 RPM.

15. A method of determining whether an oral care product inhibits hydrogen sulfide production comprising the steps:
a) mixing a solution of yeast, cysteine, and oral care product; and
b) assessing a reaction of the mixed solution to determine whether the oral care product inhibits hydrosulfide production.
